(19) United States
(12) Patent Application Publication
(10) Pub. No.: US 2011/0233059 A1
(43) Pub. Date: Sep. 29, 2011

(54) MICROFLUID SENSOR

(75) Inventors: Bernd GRÜNDIG, Leipzig (DE); Heiko Wedig, Leipzig (DE)

(73) Assignee: SensLab Gesellschaft zur Entwicklung und Herstellung bioelektrochemischer Sensoren mbH, Leipzig (DE)

(21) Appl. No.: 13/047,104

(22) Filed: Mar. 14, 2011

(30) Foreign Application Priority Data
Mar. 16, 2010 (DE) 10 2010 002 915.7

(51) Int. Cl. G01N 33/50; G01N 27/26

(52) U.S. Cl. 204/400; 422/68.1

(57) ABSTRACT

The invention relates to a microfluidic sensor which comprises a planar base sensor and a structured polymer film. The underside of the film, which faces the base sensor, comprises varyingly recessed geometric shapings and compartments which are produced, for example photolithographically, in micro injection molding, thermoforming or hot stamping processes. The microfluidic sensor according to the invention is particularly suitable for the production of biosensors in the form of single-use enzyme and affinity sensors, wherein the recessed geometries form sample collection, sample processing, incubation, buffer, mixing, reaction, reagent deposit, measurement, waste and aeration chambers, and distributing and/or connecting ducts, of which the outer peripheral contours are configured as narrow peripheral wall webs at the zero plane of the film and with a width between 50 μm and 500 μm; to which a recessed face or subsequent peripheral joining assembly with a spacing from 0.1 mm to 1.0 mm is outwardly attached.

The invention relates to a microfluidic sensor which comprises a planar base sensor and a structured polymer film. The underside of the film, which faces the base sensor, comprises varyingly recessed geometric shapings and compartments which are produced, for example, photolithographically in micro injection molding, thermoforming or hot stamping processes. The microfluidic sensor according to the invention is particularly adapted for the production of biosensors in the form of single-use enzyme and affinity sensors.

### PRIOR ART

When using diagnostic single-use sensors based on enzyme and affinity-based detection principles which enable a direct, quick and quantitative measurement for point-of-care and home-care applications, sensor structures are required which on the one hand ensure a measurement which is as correct and reproducible as possible, but on the other hand have to be producible in a cost-effective manner. These criteria necessitate a stringently reproducible liquid handling of the sample, which optionally may include sample processing steps and sample splitting as well as a multi-channel measurement of the sample. Furthermore, the sample amount required should be as small as possible.

Sensor elements which, in combination with a hand-held measuring device, make it possible to carry out a simple quantitative in situ measurement are also known as ‘single-use’ or ‘disposable’ test strips or sensors. In particular within the field of diabetes, measuring systems of this type have proven to be of use for home-care applications. During self-application, diabetes patients use glucose test strips or sensors to check their blood sugar level themselves.

Generally, part of the blood sample is conveyed by capillary force to an inner reagent surface of the sensor by the contact of a drop of capillary blood with a sample collection zone of the sensor. A specific glucose-converting enzyme, an electron acceptor and additives for stabilization and rapid wettability are deposited on the reagent surface. The enzyme may be an oxidoreductase such as glucose oxidase, glucose hydrogenase with PQQ- or FAD⁺ as a prosthetic group, or a NAD⁺-dependent dehydrogenase. For example, quinones, quinoid redox dyes or redox-active metal complexes are used as electron acceptors.

The oxidoreductase reacts with the blood glucose and transfers its electrons, which are produced during the oxidation process, to the electron acceptor which is thus reduced equivalently to the glucose concentration in the blood. This change to the redox state of the electron acceptor may be detected visually or electrochemically.

With an electrochemical or voltammetric indication of the electron acceptor, a polarization voltage is applied between a polarizable working electrode and a reference electrode and is of such a value that the reduced electron acceptor can be reliably oxidized on the anodically connected working electrode. The resultant flow of current into the outer circuit between the working electrode and the reference electrode is proportional to the sugar concentration in the blood owing to the stoichiometric conversion. A hand-held measuring device provides the necessary polarization voltage for the voltammetric measurement of the sensor, measures the signal current, detects the concentration value, shows this on the LCD and saves the measured value.

A large number of technical solutions are known for the construction of single-use sensors of this type which use an enzyme-voltammetric indication. Electrochemical-enzyme sensors for single use consist primarily of a planar base sensor with a voltammetric two- or three-electrode assembly which is arranged in a ‘measuring window’, including supply lines and electric contact faces, a reagent layer which covers the measuring window and contains the analyte-recognizing enzyme or enzyme system including the electron acceptor and additives, and a layer construction around the measuring window which supplies the sample in a rapid and defined manner.

The sample is applied either directly to the layer sequence or is drawn by capillary force effect into a capillary gap, which is arranged above the measuring window, and onto the reaction layer.

The latter technical solution was first described in EP 0 274 215 A1 and is currently applied most frequently for the production of single-use sensors for blood sugar measurement.

Adhesively bonded intermediate plastics materials layers, double-sided adhesive films or strong adhesive layers, which are applied in screen or stencil printing, are used to produce the gap. This spacer layer is applied by prior stamping out or by corresponding laminating assemblies, in such a way that they define the longitudinal sides of the measuring window, although the region of the measuring window and the end faces of the window remain uncovered. A measuring chamber is formed above the measuring window by the subsequent lamination of a cover film on the spacer layer, in such a way that an inlet and an outlet opening for the sample liquid is produced simultaneously. The thickness of the adhesive film defines the gap and the height above the measuring chamber. The length and breadth of the stamped-out opening determine the volume of the measuring chamber together with the height of the gap.

A modified technical solution provides the formation of a concave curve in the cover which produces a capillary gap after the adhesive bonding to the support. In the described technical solutions, the sample is transported onto the sensitive surface of the sensor owing to the capillary action. A large outlet opening cancels out the capillary action, in such a way that collection of the sample is thus ended. For example, variants utilize an opening in the cover or an opening in the support of the sensor as an outlet opening.

The effect of the capillary action is improved in different technical solutions by the use of hydrophilized surfaces and by the additional introduction of hydrophilic polymers and sorptive polymers or hydrophilized woven fabrics.

A microfluidic solution is described in patent DE 10211204 A1 which uses a plastics material cover film of which the face directed towards the base sensor comprises structured compartments in the form of chambers and ducts, in such a way that, after the irreversible bonding to the base sensor, a sample collection region, a measuring chamber and an air outlet gap are formed which are interconnected via ducts. The height of the chambers is determined on the one hand by the thickness of the adhesive and on the other by the depth of the structuring of the polymer film.

In the systems described above, what is common to all measuring chambers and capillary gap assemblies is that the chamber height, and thus the sample volume required, is basically determined by the thickness of the adhesive films. Furthermore, the uniform and rapid filling of the measuring chamber is dependent on the hydrophobic and hydrophilic properties of the adhesive component, which optionally has to be more strongly hydrophilized before or during the manufacturing process by an additional treatment. It should also be noted that such adhesive layers are subjected to an ageing process, as a result of which the measuring chamber geometry and the absorption behavior may change owing to shrinking processes and a loss or degradation of functionalized groups which are responsible for the hydrophilic nature of the surface of the adhesive film. Lastly, the possibilities for structuring of adhesive films, as they are currently used for single-use sensors, is limited in such a way that there is only a restricted possibility for miniaturization of microfluidic assemblies.

Further technical solutions which are primarily considered for the joining of the layer structure with a positive fit consist of adhesive bonding, welding and heat-sealing. However, the surface of the base sensor is generally provided with metal, screen-printed conductor lines or a screen-printed insulation layer which are unsuitable for these technologies, either owing to the resultant unevenness, or as a result of the material. There is also the risk that, owing to solvents in the adhesive or the necessary introduction of heat during welding or heat-sealing, the indication reagent on the measuring window already applied during the joining process will be damaged. It must be ensured that there is a sufficient distance between the joining faces and the microfluidic structures in order to prevent a thinning of the ducts through adhesive and molten material, and in order to prevent a heat-induced deformation of the plastics material defining the microfluidic region or stress formations.

For example, the use of a fiber laser is thus described, with the aid of which polycarbonate can be fused with a weld width in the region of 100 m.

Known technical solutions for the construction of immunochemical test strips or sensors which use affinity reactions are generally based on natural or synthetic filtering, particularly absorbent polymer membrane framework layers which are arranged so as to be overlapping in succession and are made of cellulose-like materials such as cellulose nitrate, cellulose ester and regenerated cellulose, or materials made of modified polyamides or modified polyethersulfone. Vertically or horizontally extending flow paths are thus formed. In addition to a separation of the sample, both the immunochemical reaction and the secondary detection reaction can take place qualitatively by visualization of a color reaction or concentration of metal nanoparticles, or quantitatively by electrochemical or visual detection. Drawbacks include the comparatively large sample volumes that are required, the limited possibilities for structuring the fluidics system, and the limited possibility for miniaturization.

With regard to the base layer a large number of technological solution approaches are known which have previously only led to commercialization for biochips of simple construction.

A possibility for producing microfluidic structures is offered by silicon technology by anisotropic etching and silicon deep-etching, and by LIGA technology in combination with micro injection molding.

Both methods are comparatively cost-intensive. Silicon technology is expedient and established in the field of R&D, in particular for the generation of complicated microfluidic structures as are required for future ‘lab-on-the-chip’ solutions, i.e. in conjunction with integrated fluidic elements such as valves, mixing chambers or micropumps and in combination with piezoelectrically or electromechanically initiated fluid transport. It could not be implemented previously for the cost-effective production of simple microfluidic disposables.

Owing to material properties, availability and potential manufacturing technologies, a solution approach with use of plastics materials appears to be more expedient if it manages to provide the microfluidic structures as structured elements at low cost and on a large scale.

Possibilities are primarily offered by micro injection molding, hot stamping and combinations thereof, as well as by newer lithographic and laser-structuring methods (EP1 225 477A1, M. F. Jensen, J. E. McCormack, B. Helbo, L. H. Christensen, T. R. Christensen, N. J. Mikkelsen, and T. Tang. “Rapid Prototyping of Polymeric Microstructures with a UV Laser, Proc. CIRP seminar on micro and nano technology”, Copenhagen, Denmark (2003)).

The technologies are adapted, above all, for rapid prototyping and for the manufacture of smaller quantities owing to the comparatively high cost.

Mass-production technologies in the case of single-use sensors therefore substantially utilize laminating methods in order to produce capillary measuring chambers above the electron assemblies and measuring windows, which methods are generated by the succession of interposed layers of adhesive and/or spacer layers and a cover film, and consequently inherently exhibit the aforementioned drawbacks.

In particular, the limited possibilities for structuring of the adhesive films also restricts the miniaturization of the fluidics systems which are also required for the further development of single-use affinity sensors, since on the one hand complex procedures are required for sample processing and for the measurement of samples with reference to a plurality of parameters, and on the other hand only a limited sample volume is available. Furthermore, in particular with passively acting microfluidic assemblies, a stringently reproducible procedure must be observed.

Owing to the compatibility with established manufacturing technologies, one solution approach with further use of laminating technology therefore appears to be worth pursuing if it manages to overcome the above-mentioned drawbacks, in such a way that microfluidic structures made of plastics material films can also be used for more complex applications in the form of single-use sensors and are suitable for mass production.

The object of the invention was therefore to provide a single-use microfluidic sensor, preferably for use as a biosensor, preferably with implementation of lamination by double-sided adhesive films, which sensor avoids the above-mentioned drawbacks, both of known single-use enzyme sensors and of single-use affinity sensors, and can be produced in a technologically cost-effective and stringently reproducible manner. Furthermore, the sensor is to be easily handled, is to absorb a minimal and well-defined sample volume, and is to make it possible to determine, for example, both substances and enzyme activity. The object of the invention is solved in accordance with independent claim 1. The dependent claims disclose preferred variants.

It has been found that a microfluidic single-use sensor formed of a base sensor 1 and a structured polymer film 2, in which the polymer film face facing towards the base sensor 1 comprises varyingly recessed geometric shapings which are arranged parallel to one another or in succession, makes it possible to achieve stringently reproducible and cost-effective properties, which are suitable for mass production, for measuring substances or enzyme activity with minimal sample volumes if the outer wall contours of geometric shapings which are recessed individually or jointly relative to the zero plane of the film 2 are configured as narrow, peripheral webs 4 at the zero plane of the film and with a width between 50 m and 500 m, and a face 5, which is recessed relative to the zero plane of the film and accommodates an adhesive film 6, or a peripheral joining assembly which follows with a spacing from 0.1 mm to 1.0 mm is attached.

The polymer film, which may consist of polycarbonate, polymethyl methacrylate, polystyrene or polyvinyl chloride and has a preferred thickness between 100 m and 250 m, is structured, for example, by hot stamping processes, a photolithographic process, laser ablation, micro injection molding or thermoforming processes.

The expression ‘structured geometries’ or ‘structured geometric shapings’ means the structuring and formation of faces and regions in the film.

The zero plane of a film is the original plane or face (=machining plane) from which structuring is conducted, i.e. from which zones are lowered or removed in a planar manner, or regions are recessed, for example by laser beam machining or hot stamping.

The depth of the structured geometric shapings on the side of the polymer film 2 facing the base sensor 1 is preferably between 0.5 m and 150 m relative to the zero plane of the film. The recessed geometric shapings may be sample collection, sample processing, incubation, buffer, mixing, reaction, reagent deposit, measurement, waste and aeration chambers, and distributing and connecting ducts which are optionally accordingly interconnected as a function of the microfluidic function to be implemented.

The outer wall webs 4, which define individual or all recessed geometric shapings, extend over the zero plane of the polymer film 2 with a width between 50 m and 500 m. The surface of the webs 4, which comprise the base sensor, is preferably planar 4a, semi-circular 4b or tapered 4c.

The depth of the inner flanks varies accordingly as a function of the depth of the geometric shapings, which is between 0.5 m and 150 m.

In a preferred embodiment a face 5, which is recessed relative to the zero plane of the film and of which the depth is identical to the outer web flank and is preferably between 20 m and 100 m, connects to the outer web flank.

This recessed face 5 which connects the wall webs serves to flushly accommodate a double-sided adhesive film 6 with a preferred thickness between 20 m and 100 m, in such a way that the structured polymer film 2 is irreversibly connected to the planar surface of the base sensor 1 with a positive and non-positive fit.

For example, a planar electrochemical sensor or a planar, visually transparent support material is used as a base sensor 1, on the measuring window 7a of which a reagent layer 8 is deposited for analyte indication. The base sensor 1 is arranged relative to the structured polymer film 2 in such a way that its measuring window 7a is arranged within a corresponding recessed, geometrical shaping which constitutes the measuring chamber 3a. The geometry and fluidic properties of the measuring chamber are determined exclusively by the structuring of the polymer film 2 and the surface properties thereof. Measuring chambers with volumes reaching into the nanoliter range can thus be stringently reproduced irrespective of the technological possibilities for structuring of the adhesive films. Since the adhesive film 6 merely performs the function of connecting the polymer film 2 to the base sensor 1 with a positive fit and, in contrast to previous technical solutions, is no longer a component of the measuring chamber and also has no specific spacer function, the structuring of the adhesive films, which is affected by tolerances, has no influence on the geometry of the measuring chamber. In particular, signs of ageing of the adhesive film such as shrinkage or the decrease in hydrophilic properties, which lead to changes in the geometry of the chamber and in the sample filling behavior, no longer play a role, in such a way that the sample volume is basically more reproducible than was previously possible above the measuring window.

In relatively complex microfluidic structures it may be advantageous, in a further variant, for a joining assembly to follow the peripheral web 4 at a distance between 0.1 mm and 1 mm, which joining assembly consists for example of two grooves 9a, 9b with an interposed joining web 10 and, in particular, joins the polymer film and the base sensor in a positive and non-positive manner by laser beam or ultrasonic welding, heat-sealing or adhesive bonding. The joining web 10 between the grooves 9a, 9b is preferably between 50 μm and 500 μm wide and has a preferred height, relative to the zero plane of the film, between −1 μm and 5 μm. The grooves 9a, 9b arranged in front of and behind said joining web are advantageously between 50 μm and 1000 μm wide and are preferably recessed, relative to the zero plane of the film, by 10 μm to 150 μm. This joining assembly is therefore advantageous, in particular, for the microfluidics system thus sealed, since any influence of the geometries arranged in a recessed manner, including reagents arranged therein, is thus avoided during the joining process. With heat generation, any damage or deformation of the microfluidic structures as well as a heat-induced deactivation of the reagent layer 8 deposited there is avoided during the welding or heat-sealing process by the groove 9a or the air gap which is arranged between the web to be fused and the recessed geometry. The two grooves 9a, 9b also intercept plastics material melts of the interposed web 10, of which the surface is melted onto by laser beam welding or heat-sealing. Similarly, the grooves 9a, 9b receive excess adhesive during the adhesive bonding of the web, in such a way that the edge regions of the microfluidics system are not coated. In order to improve the hydrophilic properties, the walls of the recessed geometric shapings in the polymer film 1 are treated with a detergent or are subjected, in a locally defined manner, to a physical plasma treatment.

In the simplest case a preferably microfluidic single-use sensor consists of a structured polymer film 2 and a planar amperometric base sensor 1, at one end of which a rectangular measuring window 7a is arranged parallel and centrally to the longitudinal axis of said base sensor. The measuring window 7a, in which an amperometric three-electrode assembly formed of a working, counter and reference electrode (11a, b, c) is arranged, is produced by the recessing of an insulation coating 7 applied to the base sensor, beneath which the conductor lines 12a-c are located between electrodes and contact faces 13a-c which are arranged at the other end of the base sensor.

Electrodes 11a-c, supply lines 12a-c and contact faces 13a-c consist of a carbon screen-printing layer or of a thin layer of noble metal which are structured photolithographically/galvanically, or by laser.

The polymer film face which faces the base sensor 1 comprises, at the measuring window 7a, a rectangular recess 3a between 5 μm and 100 μm which is produced by laser ablation, photolithography or hot stamping and of which the walls, excluding the end face pointing away from the sensor, transition into a peripheral web 4 which remains at the zero plane of the polymer film 2 and has a web width between 50 μm and 500 μm. The wall web on the opposing end face is interrupted centrally. An ‘aeration duct’ 14 with a width of 10 μm to 50 μm, a height of 25 μm to 100 μm and a length of 0.5 mm to 2.0 mm is arranged at the interruption. The aeration duct 14 opens out into a chamber 15 of large volume which comprises the air outlet openings 15a, b.

A face 5 which is recessed peripherally and relative to the zero plane of the film connects to the outer flanks of the wall webs 4, the depth of which face is identical to that of the outer web flanks and is between 20 μm to 100 μm. This recessed face 5 is used for the insertion of a double-sided adhesive film 6 with a thickness between 20 μm and 100 μm, which is stamped out in such a way that the measuring chamber 3a framed by webs fits exactly into the stamped-out part 6a. The base sensor 1 is arranged relative to the structured polymer film 2 in such a way that its measuring window 7a is located within the recessed rectangle which is framed by webs. The base face of the resultant measuring chamber 3a forms the measuring window 7a of the base sensor and the side and cover faces form the recessed rectangle. The double-sided adhesive film 6 exclusively bonds the polymer film 2 and base sensor 1 outside the measuring chamber region with a positive and non-positive fit. As a result of the adhesive bond, the surface of the peripheral wall webs 4, which points towards the base sensor and can be planar 4a, semi-circular 4b or tapered 4b, is pressed against the edge of the measuring window of the base sensor in such a way that any leakage, for example of a blood sample, out from under the web is avoided.

If a drop of blood reaches the gap-like opening of the end face of the sensor, as much sample volume as it takes to fill the measuring chamber 3a is drawn into said measuring chamber owing to the capillary force action. The air in the measuring chamber 3a displaced by the blood escapes via the aeration duct 14a into the air outlet chamber, in which atmospheric ambient pressure prevails. In the measuring chamber 3a the reagent layer is dissolved by the blood sample and the indication reaction for detecting the target analyte is enabled. The embodiment described is particularly adapted for voltammetric enzyme sensors for single use.

Owing to the very small measuring chamber volumes which can be produced with the solution according to the invention and which may be between 5.0 nl and 500 nl, filling occurs very quickly, in such a way that filling errors caused by excessively small blood droplets, lost blood droplets or limited motor capabilities of those carrying out the process themselves are drastically reduced. The measurements are thus more reliable. In particular, the very small sample volumes required are convenient for diabetics, who regularly and on a daily basis have to take a number of blood droplet samples for blood sugar measurement, and also make it possible for the measurement to be taken at alternative points of the body (alternative site testing).

A further embodiment of the microfluidic single-use sensor, which in terms of the materials used is identical to that of the embodiment above and consists of a planar base sensor 1 and a polymer film 2, is characterized in that the geometric shapings of the polymer film which are recessed relative to the zero plane of the film are a sample waste chamber 16, mixing chamber 17, affinity column 18, enzyme substrate deposit 19, measuring chambers 3b, 3c and aeration chambers 20a, b and connecting ducts 21, of which the outer peripheral contours are configured as narrow peripheral wall webs 4 at the zero plane of the film with a width between 50 μm and 500 μm, and to which a subsequent peripheral joining assembly is attached at a distance of 0.1 mm to 1.0 mm. The peripheral joining assembly consists of two grooves 9a, b with an interposed joining web 10 which is between 50 μm and 500 μm wide and is between −1 μm and 5 μm tall relative to the zero plane of the film. The grooves 9a, b are between 50 μm and 1000 μm wide and are recessed by 10 μm to 150 μm relative to the zero plane of the film.

The polymer film is fused by laser beam over the face of the peripheral joining web 10 of the joining assembly on the insulation coating 12 of the base sensor, in such a way that the recessed geometries 3ab, 16-21 are enclosed in a liquid-tight manner along the wall webs 4 surrounding them. The grooves 9a, b of the joining assembly constitute air gaps which, during the joining process, prevent any melting of adjacent structures of the recessed geometries or heat-induced deactivation of the protein-containing reagent layers 8b, c which are deposited in the measuring chambers 3b, c.

The base sensor 1 is similar to that of the first embodiment, but comprises two measuring windows 7a,b, each with a working, counter and reference electrode 11a-c and 11d-f which are arranged at the end of the sensor directly in front of the contact faces 13 a-f.

The polymer film face which faces the base sensor 1 comprises on the end face, which lies opposite the contacting faces, a sample waste chamber 16 which is arranged centrally to the longitudinal axis of the base sensor 1, comprises a volume between 3 μl and 5 μl and connects in succession to a meander-like mixing path 17 with a volume between 1 μl and 2 μl, and connecting ducts 21a. b. One of the two connecting ducts 21a leads directly, via an enzyme substrate deposit 19a with a volume between 0.5 μl and 1 μl, directly to the first measuring chamber 3b with a volume between 0.05 μl and 0.2 μl, and the other duct 21b leads to an affinity or reaction column 18 with a volume between 0.5 μl and 1 μl, which is connected via the connecting duct 21b and a further enzyme substrate deposit 19b with a volume between 0.5 μl and 1 μl to the second measuring chamber 3c. In each case the duct further leads from the two measuring chambers 3b, 3c to a sample waste chamber 20a, b which in each case comprise aeration ducts 22a, b. The sample waste chambers 22a, b have volumes between 0.5 μl and 2.0 μl.

The base sensor 1 is arranged relative to the structured polymer film in such a way that its measuring window 7b, c is in each case located within the recessed rectangle or measuring chamber 3b, 3c framed by webs.

The recessed geometries described are produced by laser ablation, photolithography or hot stamping and comprise recesses between 5 μm and 100 μm. Owing to the welded connection between the joining web 10 and the insulation layer of the substrate, the peripheral web surface, which may be planar, semi-circular or tapered, sits tightly on the insulation coating 12 of the base sensor, in such a way that the chamber walls laterally define the chamber volume in a liquid-tight manner.

With short-term contact of sample fluid with the inlet opening 16a of the sample waste chamber 16, the sample is received quickly in a capillary-force-driven defined manner and, from the sample waste chamber, fills the subsequent ducts and chambers or compartments in a passively and capillary-force-driven manner.

As the meandering assembly or mixing path 17 is filled, the antibodies, DNA portions or oligonucleotides contained therein and which are conjugated with an enzyme or a visually or electrochemically active molecule are dissolved, and are freely diffusible in the presence of the sample in the solution. The labeled molecule and the analyte are then mixed and bonded. Some of the sample, via the connecting duct 21a, reaches the enzyme substrate deposit 19a, where the enzyme substrate and co-substrate contained therein are optionally dissolved as the sample enters and a reaction begins between the marker enzyme and the enzyme substrate. The sample passes through the measuring cells and comes to a stop once the sample waste chamber 20a has been filled. The concentration of the electrochemically active reaction product formed during the enzyme reaction or the concentration of the electrochemically active marker is measured amperometrically in the first measuring chamber 3b over a defined period of time and serves as a reference and function check value. Similarly to this sandwich assay, a competitive assay can be implemented in the assembly.

The other part of the sample, via the connecting duct 21b, reaches the affinity column 18, which comprises a large surface as a result of a corresponding structuring of its walls, at which surface capture molecules such as antibodies, aptamer molecules, DNA portions or oligonucleotides are covalently immobilized. The analyte molecules, to which a marker system is bound in each case, contained in the sample are retained in the affinity column 18 owing to the affinity reaction with the capture molecules. Similarly to the other portion of the sample, the sample flowing further reaches the second measuring cell 3c via an enzyme substrate deposit 19b and also comes to a stop once the sample waste chamber 20b has been filled. The concentration of the un-bonded, remaining marker is similarly detected electrochemically. The difference between the two measurements is proportional to the analyte concentration. Instead of the electrochemical detection, a spectrophotometric, photometric or fluorimetric detection of suitable marker molecules may also be carried out respectively with use of a visually clear base sensor material. Owing to the low sample volume required, the advantageous ratio of sample volume to solid-phase surface and the simple and stringently reproducible liquid handling, this embodiment is particularly adapted for producing highly sensitive affinity sensors for single use.

Owing to the structuring in accordance with the invention of the polymer film, the base sensor and the polymer film are interconnected with a positive and non-positive fit, for example with use of a double-sided adhesive film, an adhesive or by use of a welding process, which makes it possible to achieve stringently reproducible chamber and duct geometries with volumes reaching into the lower nanoliter range irrespective of the type of positive and non-positive connection. A biosensor can thus be produced in a cost-effective manner which is suitable for mass production and is particularly adapted for the measurement of substances or enzyme activity in minimal sample volumes.

### KEY TO THE DRAWINGS

| Component | Ref. no. |
|---|---|
| Base sensor | 1 |
| Polymer film | 2 |
| Measuring chamber | 3a, b, c |
| Wall webs | 4, 4a, b, c |
| Recessed face | 5 |
| Double-sided adhesive film | 6 |
| Cut-out in the double-sided adhesive film | 6a |
| Insulation coating layer | 7 |
| Measuring window | 7a, b, c |
| Reagent layer | 8a, b |
| Grooves | 9a, b |
| Joining web | 10 |
| Working, counter and reference electrode | 11a, b, c, |
| faces | 11d, e, f |
| Supply lines | 12a-f |
| Electrical contact faces | 13a-f |
| Aeration duct | 14 |
| Air outlet chamber | 15 |
| Sample collection chamber | 16 |
| Sample collection gap | 16a |
| Meandering mixing chamber | 17 |
| Affinity column | 18 |
| Enzyme substrate deposits | 19a, b |
| Sample waste chambers | 20a, b |
| Connecting ducts | 21a, b |
| Air discharge openings | 22a, b |

The microfluidic sensor according to the invention will be described in greater detail by the embodiments and drawings below, in which:

FIG. 1 is a cross-sectional view of the microfluidic enzyme sensor through the measuring chamber 3a with a base sensor 1, polymer film 2, peripheral wall webs 4, recessed face 5, double-sided adhesive film 6, electrode faces 11a-c and reagent layer 8;

FIG. 2 is an exploded view of a microfluidic enzyme sensor with a base sensor 1, working, counter and reference electrode faces 11a-c, supply lines 12a-c, contact faces 13a-c, insulation coating 7, measuring window 7a, reagent layer 8, double-sided adhesive film 6, cut-out in the adhesive film 6a and polymer film 2 with measuring chamber 3a, peripheral wall webs 4, face 5 recessed relative to the zero plane of the film, aeration duct 14 and air outlet chamber 15;

FIG. 3 shows cross-sectional views of the measuring chamber 3a in the polymer film 2 with peripheral wall webs with a planar 4a, semi-circular 4b, and tapered 4c shaping of the face pointing towards the base sensor 2;

FIG. 4 is a cross-sectional view of the microfluidic affinity sensor through the sample waste chamber 16, with base sensor 1, polymer film 2, peripheral wall webs 4, grooves 9a, b and joining web 10;

FIG. 5 is a plan view of the microfluidic affinity sensor with a base sensor 1 and polymer film 2 with a sample waste chamber 16 and sample collection gap 16a, meandering mixing chamber 17, affinity column 18, enzyme substrate deposit 19a, b, measuring chambers 3b, c, measuring windows 7b, c, in each case with working, counter and reference electrodes 11a-c and 11d-f, electrical contact faces 14a-f, sample waste chambers 20a,b, connecting ducts 21a, b, air outlet openings 22a, b and peripherally fused joining web 10 with grooves 9a, b;

FIG. 6 is an exploded view of a microfluidic affinity sensor formed of a base sensor 1 with working, counter and reference electrode faces 11a-c and 11d-f, supply lines 13a-c and 13d-f, electrical contact faces 14a-c and 14d-f, insulation coating 7, measuring windows 7b, c and polymer film 2 with a sample waste chamber 16, and sample collection gap 16a, meandering mixing chamber 17, affinity column 18, enzyme substrate deposit 19a, b, measuring chambers 3b, c, sample waste chambers 20a, b, connecting ducts 21a, b, air outlet openings 22a, b and peripherally fused joining web 10 with grooves 9a, b.

### EXAMPLE 1

Microfluidic single-use sensor according to the invention for detecting glucose.

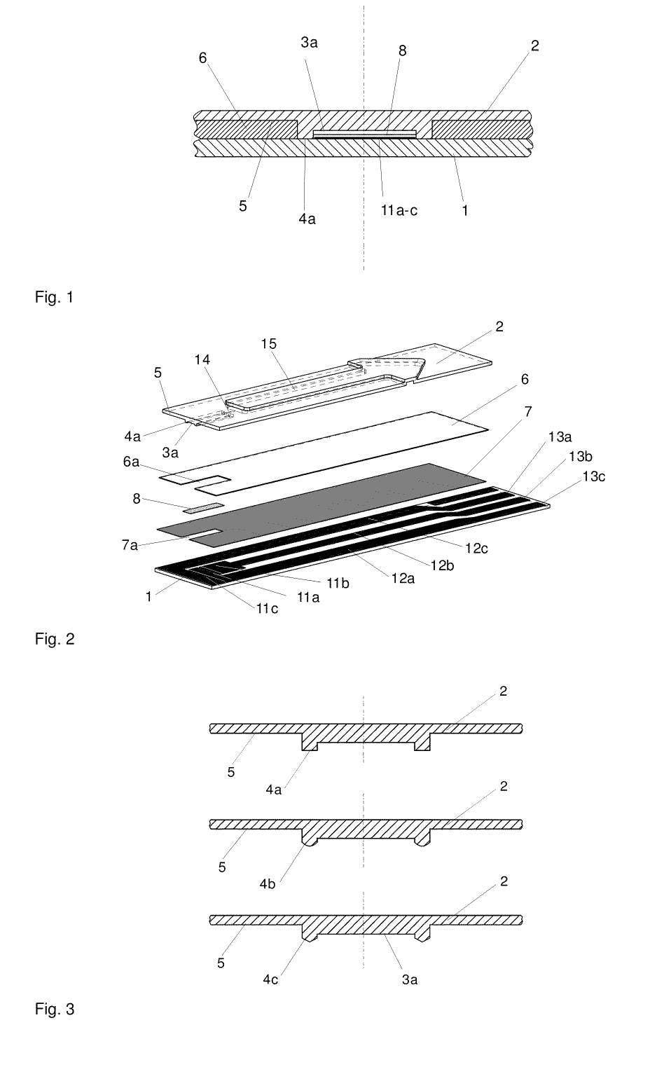

FIGS. 1 to 3 are used for purposes of explanation.

Electrode faces 11a-c, supply line paths 12a-c and contact faces 13a-c are pressed in succession in sheets of ten by screen printing onto a PET plastics material support with a thickness of 0.25 mm with the use of Acheson PE 401 carbon paste (Acheson NL) and insulation coating 12 (240 SB, ESL Europe) in order to structure an amperometric three-electrode assembly, as is shown in FIG. 2, and are then cured in each case at 70° C.

The individual faces of working, reference and counter electrodes 11a, b, c, which are arranged in succession, are 1 mm² in each case. The insulation coating 7 has a cut-out in the region of the electrode assembly which measures 1 mm×3.5 mm (w×l), in such a way that this cut-out, which constitutes the measuring window 7a, delimits the width of the electrode faces in a defined manner.

Using a dispenser, 0.3 μl of a reaction solution consisting of 2 units of glucose oxidase (Roche), 140 μg of ferricyanide (Sigma), 1.6 μg of Triton X 100 (Sigma) and 1.5 μg of microcrystalline cellulose (Aldrich) is distributed uniformly over the entire measuring window 7a as a reaction layer 8 in dispensing steps of 0.02 μl.

A polycarbonate film 0.25 mm thick is used as a polymer film and is structured by a steel male mold in a hot stamping process. The male mold produced for sheets of ten comprises elevations for a measuring chamber 3a, an aeration duct 14, a wall web 4 remaining at the zero plane and a peripherally recessed face 5 and an air outlet chamber 15. The elevation on the male mold for the recessed face 5 contacts the entire surface, apart from those faces which have just been described. Ducts and compartments with the following geometries were formed accordingly: measuring chamber 3a: 30 μm×1000 μm×3500 μm (h×w×l), aeration duct 14: 50 μm×100 μm×1 mm (h×w×l) and air outlet chamber 15: 0.250 mm×3 mm×25 mm (h×w×l). The wall webs 4 remaining at the zero plane of the film are 100 μm wide and the connecting recessed face 5 is recessed by 50 μm relative to the zero plane of the film.

A double-sided adhesive film 6 with a thickness of 50 μm is received in the region of the measuring window by stamping out cut-outs 6a measuring 1.2 mm×3.6 mm. After a correspondingly controlled laying and lamination of adhesive film 6 and stamped polymer film 2 on the base sensor 1, the measuring chamber 3a, including its wall webs 4, fits respectively into the stamped-out region of the adhesive film 6a. After 24 h the adhesive film has connected the stamped film and the base sensor to such an extent that the peripheral web 4 sits tightly on the insulation coating 12 of the base sensor. The sheet is then divided, by cutting, into ten sensors measuring 6 mm×36 mm (w×l).

The resultant sample volume required is 105 nl.

In order to carry out reproducibility tests, the sensor is connected via the contact paths 14a-c to a potentiostatic readout unit (SensLab hand-held measuring device) with a polarization voltage of +450 mV.

By contact of the end-face measuring chamber opening with a drop of blood, the measuring chamber 3a is filled in less than 0.15 s. The sample dissolves the reagent layer 8 and generates a measuring current, owing to the enzyme-electrochemical reaction, which is integrated over a time of 5 sec and is proportional to the glucose concentration contained in the blood sample. The reproducibility (VK) of ten individual measurements carried out in succession with venous, EDTA-stabilized whole blood is 1.8% with a blood glucose concentration of 4.8 mmol/L.

### EXAMPLE 2

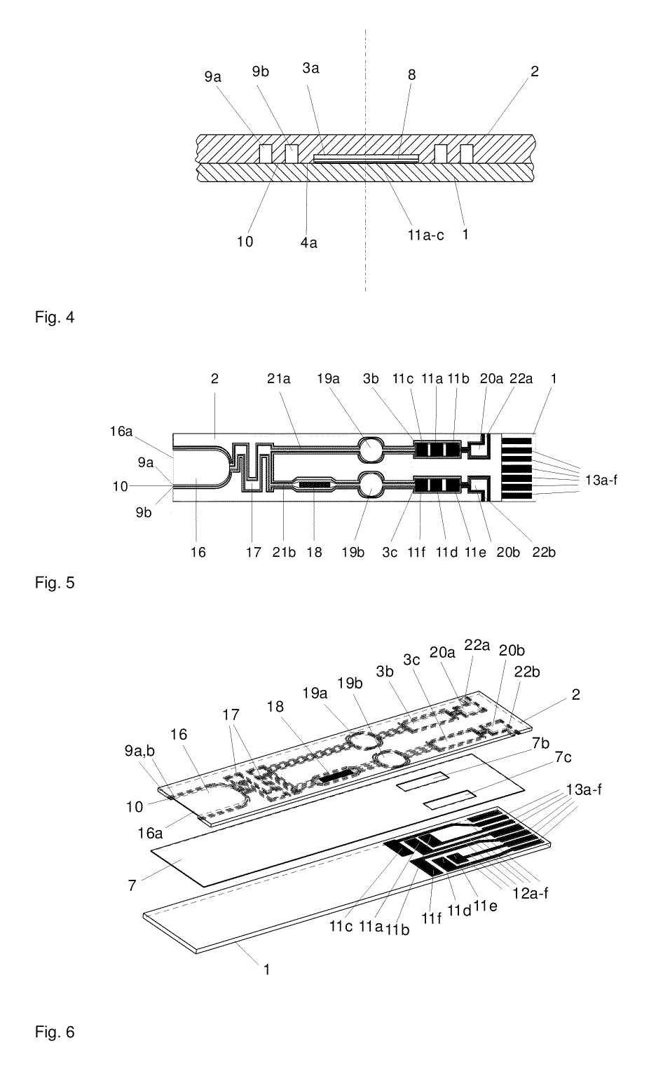

Microfluidic single-use sensor according to the invention for detecting N-acyl-histamine. FIGS. 4 to 6 are used for purposes of explanation.

Electrode faces 11a-f, pathways 12a-f and contact faces 13a-f are pressed in succession in sheets of ten by screen printing onto a PET plastics material support with a thickness of 0.35 mm with the use of Acheson PE 401 carbon paste (Acheson NL) and insulation coating (240 SB, ESL Europe) in order to structure two amperometric three-electrode assemblies, as is shown in FIG. 5, and are then cured in each case at 70° C.

The individual faces of working, reference and counter electrodes 11a-f, which are arranged in succession, are 1 mm² in each case. The insulation coating 7 has a cut-out in each case in the region of the electrode assemblies which measures 1 mm×3.5 mm (w×l) and constitutes the measuring window 7b, c. This cut-out delimits the width of the electrode faces in a defined manner.

Using a dispenser, 0.2 μl of a 0.5% Triton solution X 100 is in each case distributed uniformly over the entire measuring window 7b, c in dispensing steps of 0.02 μl and dried at 50° C. for 10 min.

A polystyrene film 0.25 mm thick is used as a polymer film and is structured by a steel male mold in a hot stamping process. The male mold produced for sheets of ten comprises elevated geometries for each sheet, as shown in FIGS. 5 and 6 for a sample waste chamber 16, a meandering mixing chamber 17, an affinity column 18, two enzyme substrate deposits 19a, b, two measuring chambers 3b, c, two sample waste chambers 20a, b, connecting ducts 21a, b, sample waste chambers 20a, b, air outlet ducts 22a, b, and a joining web 10, with grooves 9a, b, surrounding the recessed geometries 3b, c and 16-22a, b. Ducts and compartments with the following volumes were formed accordingly: sample waste chamber 16: 4 mm³, meandering mixing chamber 17: 1.5 mm³, affinity reaction chamber 18: 1.5 mm³, each enzyme substrate deposit chamber 19a, b: 0.15 mm³, each measuring chamber 3b, c: 0.2 mm³, each sample waste chamber 20: 1 mm³.

1.5 μl of a 0.5% Triton solution X 100 are introduced into the sample waste chamber, 17 β-galactosidase-histamine conjugate (0.2 μg/ml, β-galactosidase, Calbiochem) is introduced into the meandering mixing chamber, and 0.5 μl of p-aminophenyl-β-D-galactoside solution (0.5 mM with 0.5% gelatin) was introduced into each of the enzyme substrate deposit chambers 19a, b. After interposed drying at 30° C. for 40 min, 1.2 μl of anti-rabbit antiserum (Ziege), which is absorbently bonded to the plastics material surface over 30 min at 30° C., is added to the affinity column 18. The affinity column is then rinsed carefully with a blocking buffer and dried again.

The polymer film 2 prepared in this manner is fixed over the base sensor in such a way that the two measuring windows 12b, c are each positioned beneath the measuring chambers 3b, c and the sample waste chamber 16 terminates at the end face of the base sensor 1. The peripheral joining web 10 is fused to the insulation layer 8 of the base sensor by laser beam, so these are liquid-tight as far as apart from the sample inlet gap 16a and the air outlet openings 22a, b of the sample waste chambers 20a,b. As a result of the welded connection, the peripheral wall webs 4 also sit tightly, along the outer contours of the chambers and ducts, on the insulation coating 7 of the base sensor, in such a way that any leakage of the sample out from beneath the wall webs is prevented.

In order to carry out the histamine determination the two three-electrode assemblies of the measuring window are each connected via the contact paths 13a-c and 13d-f to a potentiostatic readout unit (SensLab hand-held measuring device) with a polarization voltage of +200 mV vs. internal reference electrode.

The sample is collected in a brisk and defined manner by contact of the sample with the sample collection gap 16a of the sample waste chamber 16 and this leads to the passive filling, driven by capillary force, of the subsequent ducts and chambers or compartments.

As the meandering mixing path 17 becomes full, the acetyl histamine which is contained therein, is labeled with β-galactosidase and is freely diffusible in the presence of the sample in the solution is dissolved. The labeled acetyl histamine and the acetyl histamine in the sample are mixed. Some of the sample, via the first connecting duct 21a, reaches the enzyme substrate deposit 19a, where the p-aminophenyl-β-D-galactosidase contained therein is dissolved in a delayed manner owing to the gelatin layer and a reaction by β-galactosidase (the marker enzyme of the conjugate) is begun. The sample passes through the measuring cells and comes to a stop once the sample waste chamber 20a is full. The concentration of the electrochemically active p-aminophenol which is cleaved during the enzyme hydrolysis is measured amperometrically in the first measuring chamber 3a at the three-electrode assembly of the first measuring window 7b over a defined period of time and serves as a reference and function check value.

The other part of the sample, via the connecting duct 21b, reaches the affinity column 18, which comprises a large surface as a result of its structuring and on which the capture antibodies are absorbently immobilized. The acetyl-derivatized histamine contained in the sample enters into a competitive reaction with the β-galactosidase-acetyl-histamine conjugate around the binding sites of the antibody layer immobilized in the affinity reaction chamber 18.

The more acetyl-histamine there is contained in the sample, the less conjugate is bonded. The sample flowing further, and with it the un-bonded β-galactosidase-histamine conjugate, reaches the second enzyme substrate deposit 19b via the connecting channel 21b. The p-aminophenyl-β-D-galactoside present is dissolved and enzyme hydrolysis to form p-aminophenol is begun. The sample directly enters the second measuring chamber 3c and comes to a stop once the second sample waste chamber 21b is full.

The β-galactosidase of the conjugate, which could not bond in the affinity column 18 and reaches the second measuring cell 3b together with the sample flow, forms the electrochemically active p-aminophenol, similarly to the reference duct, which is detected amperometrically via the three-electrode assembly of the second measuring window 7c in the measuring chamber 3c. The difference between measurements in the measuring chamber 3c and 3b is proportional to the analyte concentration.

1. A microfluidic sensor which comprises a base sensor (1) and a structured polymer film (2), the underside of the polymer film, which faces the base sensor (1) and of which the face forms the zero plane of the film, comprising varyingly recessed geometric shapings relative to the zero plane of the film which are arranged parallel or in succession and form sample collection, sample processing, incubation, buffer, mixing, reaction, reagent deposit, measurement, waste and aeration chambers, and distributing and/or connecting ducts (3, 15, 16, 17, 18, 19, 20, 21), characterized in that the outer peripheral contours of these recessed geometrical shapings are configured as narrow peripheral wall webs (4) at the zero plane of the film (2) and with a width between 50 μm and 500 μm; to which a recessed face (5) or subsequent peripheral joining assembly spaced from 0.1 mm to 1.0 mm is outwardly attached, which connects the base sensor (1) and polymer film (2).

2. The microfluidic sensor according to claim 1, characterized in that the film (2) has a thickness between 100 μm and 250 μm.

3. The microfluidic sensor according to either claim 1 or claim 2, characterized in that the geometric shapings which are recessed relative to the zero plane of the film have depths between 0.5 m and 150 m and are optionally interconnected.

4. The microfluidic sensor according to any one of claims 1 to 3, characterized in that the faces of the peripheral wall webs (4a, b, c) pointing towards the base sensor (1) are planar, semi-circular or tapered.

5. The microfluidic sensor according to any one of claims 1 to 4, characterized in that connecting faces (5) which are recessed relative to the zero plane of the film (2) have a depth between 20 m and 100 m and flushly accommodate a double-sided adhesive film (6) with a thickness between 20 m and 100 m.

6. The microfluidic sensor according to any one of claims 1 to 5, characterized in that the peripheral joining assembly (10) consists of two grooves (9a, b) with an interposed web (10).

7. The microfluidic sensor according to claim 6, characterized in that the web (10) between the grooves (9a, b) is 50 m to 500 m wide and is between −1 m and 5 m tall relative to the zero plane of the film (2).

8. The microfluidic sensor according to claim 6 and claim 7, characterized in that the grooves (9a, b) are between 50 m and 1000 m wide and are recessed by 10 m to 150 m relative to the zero plane of the film.

9. The microfluidic sensor according to any one of claims 1 to 8, characterized in that a planar electrochemical sensor or a planar visually transparent support material acts as the base sensor (1), and a film made of polycarbonate, polyamide, polystyrene or an acrylate acts as the polymer film (2).

10. The microfluidic sensor according to any one of claims 1 to 9, characterized in that the polymer film (2) is structured by hot stamping processes, by a photolithographic process, laser ablation, micro injection molding or thermoforming processes.
